(19) United States
(12) Patent Application Publication
(10) Pub. No.: US 2009/0131881 A1
(43) Pub. Date: May 21, 2009

(54) CANNULA

(76) Inventor: William Anthony Frisella, Jr., St. Louis, MO (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 11/944,154

(22) Filed: Nov. 21, 2007

(51) Int. Cl. A61M 25/00

(52) U.S. Cl. 604/264

(57) ABSTRACT

A cannula including an inner structure, an outer structure, and a flexible portion. The outer structure is disposed about the inner structure and movable with respect to the inner structure. The flexible portion is attached to the inner structure at an end thereof and attached to the outer structure at an end thereof. The flexible portion is adapted to be stretched from a first smaller diameter when the outer structure is in a first position to a larger diameter when the outer structure is in a second position.

### FIELD OF THE INVENTION

The present invention is related to cannulas. More particularly, the present invention relates to an improved cannula with a retractable anchoring system.

### BACKGROUND OF THE INVENTION

Endoscopic techniques allow surgical procedures to be performed in a minimally invasive manner, thereby avoiding many of the complications associated with traditional, or open, surgery. Typically, a small puncture is made in the skin and one or more instruments are inserted by way of cannulas for access to the area of interest. Cannula devices are typically tubular in shape and can be of various sizes.

A problem inherent in the use of surgical cannulas is the risk of dislodgement from the body cavity opening. In addition, soft tissue can obscure vision or interfere with the passage or use of instruments. A surgical cannula may be dislodged by manipulation of surgical instruments through the cannula during use. When a cannula becomes dislodged, it must be re-inserted. A common problem is that of having to re-insert the device back through the previously made tract through the soft tissues to regain access to the body cavity. This becomes increasingly difficult with each dislodgement episode. Dislodgement of a cannula results in increased operative time and increased surgical trauma with the necessity of re-inserting the cannula. There are several cannula devices available that attempt to decrease the risk of dislodgement. Complex devices preclude their common employment as a result of 1) their prohibitive price and 2) the increased risk of malfunction inherent in their more complex construction. Cannulas incorporating additional stabilizing elements are often inadequate and are not commonly employed.

Previous designs suffer a number of deficiencies. For example, U.S. Pat. No. 4,608,965 describes an endoscope having a single soft plastic cylindrical tube with one end being formed having a smaller end diameter which is forced over the probe portion of an endoscope, and a portion of the soft plastic cylindrical tube adjacent the end having the smaller diameter is formed having longitudinal slits forming flexible strips. U.S. Pat. No. 5,197,971 describes a retractor having a complex mechanical expanding portion for expanding against sub-surface tissues when the retractor is in use, and describes a fluid-operated expanding portion.

Other devices are provided with serrations, protuberances or threads at their tips, in order to stabilize them in an anatomical cavity. For example, U.S. Pat. No. 7,125,411 provides a flexible coil to be secured to the cannula device after being inserted through the puncture opening. Similarly, U.S. Pat. No. 6,808,494 describes an accordion-like tube which expands against and grips the side walls of an incision. Another example is U.S. Pat. No. 6,542,283 which utilizes a balloon anchor for stabilization of the cannula device. U.S. Pat. No. 5,637,097 to Yoon describes a penetrating instrument having an expandable anchoring portion having a membrane, wherein the expandable portion is biased in the expanded position by means of forming the expandable portion of a resilient spring material or a material having shape memory. Other related art includes U.S. Pat. No. 5,002,557, and U.S. Pat. No. 5,882,340. The teachings of these patents are hereby incorporated by reference.

None of the available designs, however, are effective in maintaining the cannula in proper position while providing a simple two-tube device for deploying and retracting an anchoring flange. For example, Yoon provides a bias means for employing the expandable portion which is ineffective for preventing the retraction of the device while using the cannula to manipulate the cavity. A surgeon may use the cannula to expand the cavity for operation by pulling the cannula, which would cause the cannula taught by Yoon to retract—a problem which is overcome by the present invention.

In addition, none of the available designs provide the above benefits while preventing fluid and/or tissue from entering the expandable portion of the cannula cavity, and becoming caught by portions of these devices. Furthermore, none of the available designs provide the above benefits while providing a tool for manipulating the operation cavity. Also, none of the available designs provide the above benefits while providing an outer flange limiting the entry distance of the distal portion of the cannula into the cavity. Furthermore, none of the available designs provide the above benefits while providing a simple mechanism for deploying the stabilizing portion of the cannula in a fixed position.

### SUMMARY OF THE INVENTION

The present invention provides a cannula comprising an inner structure, an outer structure, and a flexible portion. The outer structure is disposed about the inner structure and movable with respect to the inner structure. The flexible portion is attached to the inner structure at an end thereof and attached to the outer structure at an end thereof. The flexible portion is adapted to be stretched from a first smaller diameter when the outer structure is in a first position to a larger diameter when the outer structure is in a second position.

### BRIEF DESCRIPTION OF THE DRAWINGS

FIG. 1 is a perspective view of a cannula in a first position according to an embodiment of the present invention;

FIG. 2 is a perspective view of a cannula in a second position according to an embodiment of the present invention;

FIGS. 3-6 illustrate the use of a cannula according to an embodiment of the present invention;

FIG. 7 is a partial section side view of a cannula lock according to an embodiment of the present invention;

FIGS. 8A and 8B are a partial section side view and a top view of a cannula lock according to an embodiment of the present invention; and

FIG. 9 is a perspective view of a cannula having an external limiter according to an embodiment of the present invention.

### DETAILED DESCRIPTION OF THE INVENTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated.

The preferred embodiment, described below, relates to a cannula having an improved anchoring system. The improved cannula is simple to operate and inexpensive to manufacture. The cannula further has the ability to remain in position while a surgical procedure is performed to prevent waste of time from having to reinsert the cannula and to prevent unnecessary damage to the patient's tissue due to unnecessary movement and reinsertion of the cannula.

In that regard and referring to FIG. 1, the inventive cannula 10 of the preferred embodiment comprises a hollow inner structure 12 and a hollow outer structure 14 that are preferably cylindrical. The outer structure 14 is disposed about the inner structure 12 and movable with respect to one another along a common central axis. The inner structure 12 may optionally comprise an enlarged end 16. An opening of the enlarged end 16 may be covered with a diaphragm 18 through which surgical instruments may be inserted. Diaphragms 18 covering an end of a cannula are well known in the art and will not be further described herein.

The outer structure 14, as stated above, disposed about the inner structure 16 and is easily movable with respect to the inner structure 12. Attached to the outer structure 14 is a flexible portion 20 that has the ability to resiliently stretch and deform. The flexible portion 20 is preferably made from a polymeric material. The flexible portion 20 comprises a first attachment portion 22, second attachment portion 26 and a hinge point 24 between the attachment portions 22 and 26.

The first attachment portion 22 comprises the interface where the flexible portion 20 attaches to the outer structure 14. The outer structure 14 may itself be made from the same flexible material or maybe made from a more rigid material and adhered to the flexible portion 20 by a solvent weld, friction weld or other suitable attachment method.

The second attachment portion 26 attaches the inner structure 12 to the flexible portion 20. Again, the flexible portion 20 and the inner structure 12 may be made from the same material or attached by a suitable attachment method.

The hinge point 24 is a weakened circle around the circumference (if the flexible portion is cylindrical) along which greater flexibility in the flexible portion 20 is experienced causing the circle around the circumference to act as a living hinge, as further described below. The hinge point 24 may optionally be made by pre-stretching or pre-stressing a portion of the flexible portion 20 or by creating a score line in the flexible portion, for example. Regardless of the method chosen, which will be dependent upon the material chosen for the flexible portion 20, the flexible portion 20 will have the ability to behave as a living hinge at the hinge point 24.

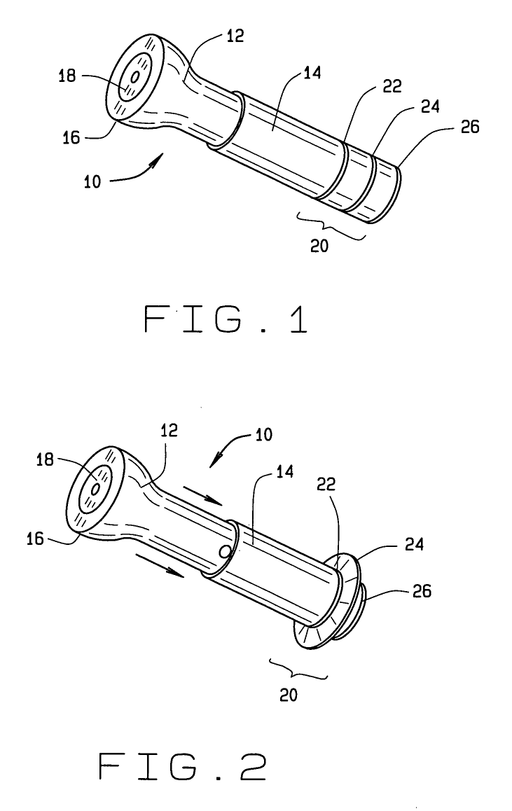

Referring to FIG. 2, when the outer structure 14 is moved toward the attachment portion 26, the flexible portion 20 expands and folds at the hinge point 24 to create an enlarged flange having a diameter greater than the flexible portion 20 before movement of the outer structure 14. Near the attachment points 22 and 26, the flexible portion 20 also folds and acts as a living hinge to allow for the outward movement of the flexible portion 20.

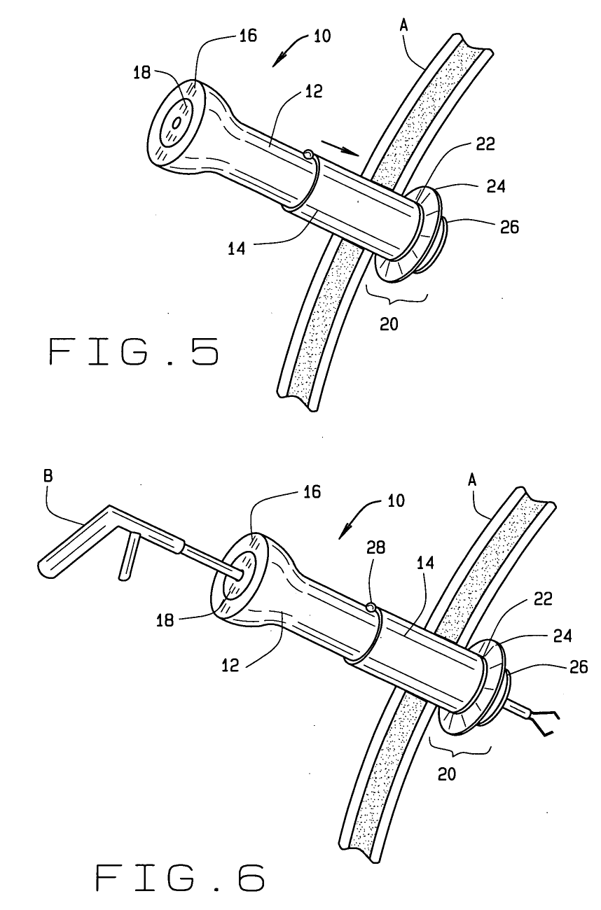

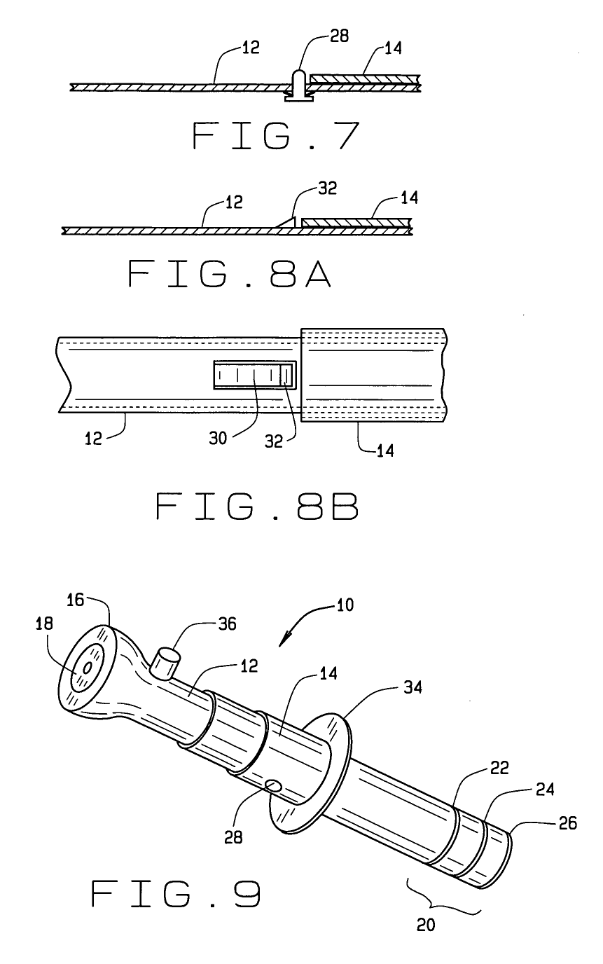

The cannula 10 further provides a lock mechanism for locking the outer structure 14 with respect to the inner structure 12 in the position of FIG. 2. By means of example and not by limitation, several known lock structures may be used, such as in FIG. 6 where a spring-loaded detent 28 rises when then outer structure 14 passes from above to prevent return of the outer structure 14 to the initial position. The detent 28 may optionally pass through a bore in the outer structure (FIG. 9). Alternatively in FIG. 7, a resilient tab 30 formed from the inner structure 12 having a raised barb 32 could be implemented to form the lock mechanism. One of ordinary skill in the art would recognize a myriad of lock structures that could be used to maintain the inner structure 12 in a fixed position with respect to the outer structure 14, and it is the concept the lock structure that is inventive and claimed below and not any specific lock structure unless a specific lock structure is explicitly claimed below.

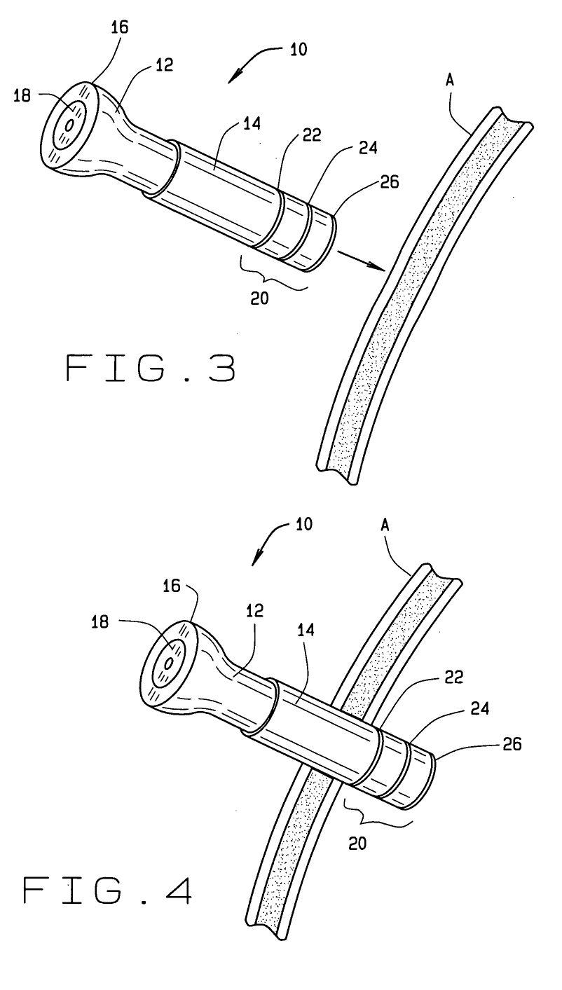

The cannula 10 is used as shown in FIGS. 3-6. In a first step (FIG. 3) the cannula 10 is brought into position next to a patient's skin A. A puncture is made, commonly with a trocar (not shown), through the skin A by inserting the trocar through the cannula. In a second step (FIG. 4), the cannula 10 is inserted through the puncture in the skin such that the flexible portion is within the patient's body. In a third step (FIG. 5), the outer structure 14 is moved coaxially with the inner structure 12 to expand the flexible portion 20 to form a flange and lock the outer structure 14 with respect to the inner structure 12. In a fourth step (FIG. 6), the cannula 10 is secured within the body such that it will not accidentally become dislodged while the surgeon uses instruments B through the cannula 10 to perform the operation. To remove the cannula 10, the surgeon merely disengages the lock mechanism to return the cannula 10 to the configuration of FIG. 4 and removes the cannula 10 from the body.

As shown in FIG. 9, the cannula 10 may include an external limiter 34 for limiting the entry distance of the cannula 10 into the opening in the patient's body. The external limiter 34 can also be used to provide further stability for the cannula 10 when the flexible portion 20 is in the large diameter state. Accordingly, the external limiter 34 can be connected to the outer structure in a fixed position. Alternatively, the external limiter 34 can movable with respect to the outer structure

The cannula 10 may also optionally include a side port 36 for the introduction or removal of fluid, as is known in the art.

The above examples show that the invention, as defined by the claims, has far ranging application and should not be limited merely to the embodiments shown and described in detail. Instead the invention should be limited only to the explicit words of the claims, and the claims should not be arbitrarily limited to embodiments shown in the specification. The scope of protection is only limited by the scope of the accompanying claims, and the Examiner should examine the claims on that basis.

I claim:

1. A cannula comprising:
an inner structure;
an outer structure disposed about the inner structure and movable with respect to the inner structure; and
a flexible portion attached to the inner structure at an end thereof and attached to the outer structure at an end thereof, the flexible portion adapted to be expanded from a first smaller diameter when the outer structure is in a first position to a larger diameter when the outer structure is in a second position.

2. The cannula of claim 1 wherein the flexible portion comprises hinge point comprising a score line.

3. The cannula of claim 1 wherein the flexible portion comprises hinge point comprising a pre-stressed portion.

4. The cannula of claim 1 further comprising a lock to hold the outer structure in a fixed position with respect to inner structure.

5. The cannula of claim 4 wherein the lock comprises a spring loaded detent.

6. The cannula of claim 4 wherein the lock comprises a resilient tab formed in the inner structure.

7. The cannula of claim 1 further comprising an enlarged end.

8. The cannula of claim 7 further comprising a diaphragm at the enlarged end.

9. The cannula of claim 1 further comprising an external limiter for limiting the depth the cannula may be inserted within a body.

10. A cannula comprising:
an inner structure;
an outer structure disposed about the inner structure and movable with respect to the inner structure;
a flexible portion attached to the inner structure at an end thereof and attached to the outer structure at an end thereof, the flexible portion adapted to be expanded from a first smaller diameter when the outer structure is in a first position to a larger diameter when the outer structure is in a second position; and
a lock to hold the outer structure in a fixed position with respect to inner structure.

11. The cannula of claim 10 wherein the flexible portion comprises hinge point comprising a score line.

12. The cannula of claim 10 wherein the flexible portion comprises hinge point comprising a pre-stressed portion.

13. The cannula of claim 10 wherein the lock comprises a spring loaded detent.

14. The cannula of claim 10 wherein the lock comprises a resilient tab formed in the inner structure.

15. The cannula of claim 10 further comprising an enlarged end.

16. The cannula of claim 10 further comprising a diaphragm at the enlarged end.

17. The cannula of claim 10 further comprising an external limiter for limiting the depth the cannula may be inserted within a body.

18. A method of using a cannula comprising the steps of:
providing a cannula having an inner structure, an outer structure disposed about the inner structure and a flexible portion attached to the inner structure at an end thereof and attached to the outer structure at an end thereof;
inserting the cannula into an opening in a patient's skin until the flexible portion is within the patient's body;
moving the outer structure from a first position with respect to the inner structure to a second position with respect to the inner structure to stretch the flexible portion to a larger diameter, the enlarged diameter disposed within a patient's body and under his skin to anchor the cannula within the opening;
using the cannula to perform a surgical operation;
moving the outer structure back to the first position from the second position to reduce size of the flexible portion from the larger diameter to the smaller diameter; and
removing the cannula from the patient's body.

19. The method of claim 18 further comprising the step of locking the outer structure in the second position while performing the step of using the cannula to perform a surgical operation.
